(19) United States
(12) Patent Application Publication
(10) Pub. No.: US 2026/0294944 A1
(43) Pub. Date: Oct. 1, 2026

(54) COMPOSITION FOR USE IN A SUBJECT

(71) Applicant: SOCIÉTÉ DES PRODUITS NESTLÉ S.A., Vevey (CH)

(72) Inventors: Fabio MAINARDI, Lausanne (CH); Purva RAJHANS, Lausanne (CH); Nora SCHNEIDER, Les Monts-de-Corsier (CH)

(21) Appl. No.: 19/477,696

(22) PCT Filed: Apr. 26, 2024
(86) PCT No.: PCT/EP2024/061678

§ 371 Date: Oct. 22, 2025

(30) Foreign Application Priority Data
Apr. 28, 2023 (EP) 23170830.6

(51) Int. Cl. A61K 31/702; A23L 33/00; A61P 25/28

(52) U.S. Cl. CPC A61K 31/702; A23L 33/40; A61P 25/28

(57) ABSTRACT

The present invention relates to one or more human milk oligosaccharides (HMOs) for use in supporting brain development and/or cognitive development in a subject. In particular, the one or more HMOs comprise or consist of: fucosyllactose (3FL), lacto-N-fucopentaose II (LNFPII), and/or lacto-N-fucopentaose V (LNFPV), and/or any combination thereof. The brain and/or cognitive development comprises language development. Furthermore, the present invention relates to method for supporting brain development and/or cognitive development in a subject.

### FIELD OF THE INVENTION

The present invention relates to one or more human milk oligosaccharides (HMOs) for use in supporting brain development and/or cognitive development in a subject. In particular, the one or more HMOs comprise or consist of: fucosyllactose (3FL), lacto-N-fucopentaose II (LNFPII), and/or lacto-N-fucopentaose V (LNFPV), and any combination thereof. The brain and/or cognitive development comprises language development. Furthermore, the present invention relates to method for supporting brain development and/or cognitive development in a subject.

### BACKGROUND OF THE INVENTION

Infancy is a critical period for brain maturation, and for the development of cognitive functions and socio-emotional skills. Brain development occurs during the aging of a child and is most essential in early childhood. It places exceptionally high demands on the supply of key nutrients to a child. Failure to meet these nutrient demands during this crucial period may result in sub-optimal neurodevelopment, in particular brain development. Milk is the main source of nutrients and energy during the first three months of life, therefore providing the building blocks for the underlying biochemical processes, including myelination. Breast milk has been advocated by the WHO as the exclusive source of nutrients for infants during the first 6 months of life (WHO). Exclusive breastfeeding has been associated to better cognitive performance (Belfort et al. JAMA Pediatr. 2013 September; 167 (9):836-44).

Human milk oligosaccharides (HMOs) form the third predominant component of human milk, after fat and lactose, with an estimated concentration ranging between 5 and 15 g/L. Although largely not digestible by the infant's gut, HMOs have a range of known physiological functions, including the shaping of the gut microbiota (Šuligoj T et al. Nutrients. 2020 Sep. 13; 12(9):2808), and cytokine production (Eiwegger et al. Pediatr Res. 2004 October; 56(4):536-40).

More than 150 HMOs have been identified and are unique to human milk. The proportions of concentrations of HMOs in breast milk vary among individuals and depend in particular on the expression of α1-2-fucosyltransferase (FUT2) and α1-3,4-fucosyltransferase (FUT3) genes which determine the secretor (Se) and Lewis (Le) status. 2′-fucosyllactose (2′FL) is absent in the milk of non-secretor women, while is the most abundant HMO in the milk of secretors.

Breastmilk composition is known to evolve during the lactation period (Samuel et al); in particular, the total concentration of HMOs decreases (Samuel et al. Scientific Reports 2019; 9(1):11767), with most of the individual HMOs decreasing as well, with the notable exception of 3FL.

Associations of individual HMOs with cognitive development have been reported in several cohorts (Berger et al. Nutrients 2023; 15(3):719). These associations are typically cross-sectional, based on the correlation between the level of an HMO and some cognitive outcome at two fixed time points. Furthermore, the findings that infant exposed to higher levels of the sialylated HMO 3′SL had higher language scores in infants older than 12 months of age are specific to infants of mothers with blood type A (A-tetra+) and mothers classified as secretors (SE+) (Cho et al. Am. J. Clin. Nutr. 2021; 114(2):588-597).

The potential interactions between HMOs and gut microbiota have been implicated since HMOs are largely metabolized in the intestinal tract. Gut microbiota composition and particular HMOs (e.g., 2-FL) have shown to be independently associated with infant cognition. For example, using chronic oral administration of 2′FL to rodents, Vazquez et al. demonstrated that the treated animals exhibit significantly better learning and working memory function (Vazquez et al., 2015, Journal of nutritional biochemistry, 26(5):455-465). However, there is a paucity of data on the potential effects of other HMOs as well as the interactions between gut microbiota and HMOs on brain functional development in infancy and early childhood.

EP 2117355 relates to use of a composition comprising non-digestible saccharide selected from the group consisting of galactooligosaccharides (GOS), fructooligosaccharides (FOS) and fructopolysaccharides for the manufacture of a composition for preventing a decline in or improving one of more of (i) language skills, (ii) communication skills, (iii) social skills and/or (iv) reading skills. This reference reported that composition containing prebiotic oligosaccharides (FOS/GOS) reduces the level of pathogenic Clostridium bacteria and thus is indicative of a positive effect on language skills, communication skills, social skills and/or reading skills.

Thus, there is a need for new nutritional interventions to support healthy cognitive development, for example in infants. There is a need to deliver such benefit in a manner that does not induce side effects and/or in a manner that is easy to deliver, and well accepted by the parents or health care practitioners. There is also a need to deliver such benefits in a manner that keeps the cost of such delivery reasonable and affordable by most.

### SUMMARY OF THE INVENTION

The first three years of life, when the brain is maturing, is the most intensive period for acquiring speech and language skills. The language development that occurs in early childhood lays important foundation for communicating and learning for the rest of child's life. Trouble in understanding what other says (receptive language) or difficulty sharing their thoughts (expressive language) may be considered as a language disorder. Developmental language disorder (DLD) is a language disorder that delays the mastery of language skills. Although children may vary in their development if speech and language skills, in order to avoid delays in language development that could lead to DLD, it is important that the infants and young children follow a natural progression or timetable for mastering the skills of language.

The inventors have surprisingly found that specific fucosyllated human milk oligosaccharides (HMOs), namely 3-fucosyllactose (3FL), lacto-N-fucopentaose II (LNFPII), lacto-N-fucopentaose V (LNFPV), are significantly associated with brain and cognitive development, and in particular in the development of language in infants and young children. Interestingly, the inventors observed significant association between language development at 12 months and levels of 3FL, LNFPII and LNFPV at 2-5 weeks. Moreover, 3FL levels at 6 weeks and 6 months is also significantly associated with language development at 12 months. Without to be bound by the theory, the one or more HMOs significantly identified in the present invention are advantageously supporting brain and cognitive development in infants and young children, more particularly they support language development in infants and young children.

An object of the present invention is to improve the state of the art and to provide an new nutritional intervention, or at least to provide a useful alternative, to overcome at least some of the inconveniences described above

The object of the present invention is achieved by the subject matter of the independent claims. The dependent claims further develop the idea of the present invention.

Accordingly, the present invention provides in a first aspect one or more human milk oligosaccharides (HMOs) for use in supporting brain development and/or cognitive development

In another aspect, the present invention provides use of one or more human milk oligosaccharides (HMOs) for use in supporting brain development and/or cognitive development in a subject.

In another aspect, the present invention provides a method for supporting brain development and/or cognitive development in a subject.

Any suitable HMOs described therein may be used. Suitably, the one or more HMOs comprise or consist of fucosylated HMOs. Suitably, the one or more HMOs comprise or consist of: 3-fucosyllactose (3FL), lacto-N-fucopentaose II (LNFPII), lacto-N-fucopentaose V (LNFPV), and/or any combination thereof.

The one or more HMOs may be administered by any suitable means and in any suitable amounts. Suitably, the one or more HMOs are administered by oral administration. Suitably, the one or more HMOs are administered separately, simultaneously or sequentially. In preferred embodiments, the one or more HMOs are administered simultaneously. Suitably, 3FL is administered to the subject in a total amount of from about 3.5 mg/L to about 5,000 mg/L, LNFPII is administered to the subject in an amount of from about 16 mg/L to about 2,400 mg/L and LNFPV is administered to the subject in an amount of from about 9 mg/L to about 450 mg/L.

The one or more HMOs may be provided in any suitable form. Suitably, the one or more HMOs are in the form of a nutritional composition. In some embodiments, the nutritional composition is an infant formula, a follow-on formula, or a growing-up milk.

In preferred embodiments, the nutritional composition is an infant formula. Suitably, the starter infant formula, follow-on formula, and growing-up milk each comprise from about 60 kcal/100 mL to about 80 kcal/100 mL, protein in an amount of from about 1.5 g/100 kcal to about 2.5 g/100 kcal, carbohydrate in an amount of from about 8 g/100 kcal to about 15 g/100 kcal, and lipids in an amount of from about 3 g/100 kcal to about 8 g/100 kcal.

The nutritional composition may comprise the one or more HMOs in any suitable amount. Suitably, the nutritional composition comprises a combination of fucosylated HMOs in a total amount of from about 0.01 g/day to about 5 g/day. Suitably, the nutritional composition comprises 3-fucosyllactose (3FL) in an amount of from about 3.5 mg/L to about 5,000 mg/L, lacto-N-fucopentaose II (LNFPII) in an amount of from about 16 mg/L to about 2,400 mg/L and lacto-N-fucopentaose V (LNFPV) in an amount of from about 9 mg/L to about 450 mg/L.

The subject may be any suitable subject. The subject may be a human subject. In some embodiments, the subject is an infant, a toddler or a young child. In preferred embodiments, the subject is an infant. In some embodiments, the subject is aged about 12 months or younger, about 11 months or younger, about 10 months or younger, or about 9 months or younger, about 6 months or younger, about 3 months or younger, or about 1 months or younger.

The one or more HMOs may improve the subject's language development. The language development may be determined by a Bayley Scales of Infant and Toddler Development (BSID-IV).

### BRIEF DESCRIPTION OF THE DRAWINGS

FIG. 1—Correlations between concentrations of 3FL (mg/ml) and Bayley Scales of Infant and Development IV (BDSI-IV) at the three time points of lactation: (A) at 2-5 weeks, (B) at 6 weeks and (C) at 3 months. Language development is assessed at 12 months. Solid lines are regression lines, with 95% confidence bands.

FIG. 2—Correlations between LNFPII (mg/ml) and Bayley Scales of Infant and Development IV (BDSI-IV) at 2-5 weeks. Language development is assessed at 12 months. Solid lines are regression lines, with 95% confidence bands.

FIG. 3—Correlations between LNFPV (mg/ml) and Bayley Scales of Infant and Development IV (BDSI-IV) at the three time points of lactation: left panel 2-5 weeks, middle panel at 6 weeks and right panel at 3 months. Language development is assessed at 12 months. Solid lines are regression lines, with 95% confidence bands.

### DETAILED DESCRIPTION OF THE INVENTION

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

As used in this specification, the words “comprises”, “comprising”, and similar words, are not to be interpreted in an exclusive or exhaustive sense. In other words, they are intended to mean “including, but not limited to”. The terms “comprises”, “comprising”, and similar words also include the term “consisting of”.

The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The term “subject” as used therein refers to a mammal and more particularly a human. In one embodiment, the subject is an infant, a toddler or a young child.

The term “infant” means a child under the age of 12 months. The term infant includes both infant born at term and infant born preterm.

The term “young child” or “toddler” means a child aged between one and three years.

The term “child” means a child aged between one and 6 years, including toddlers and pre-school children.

The Bayley Scales of Infant and Child Development Fourth Edition (BSID-IV) is a standard series of measurement used to assess the development of infants and toddlers from 1-42 months in five domains—cognition, motor, language, socio-emotional and adaptive behavior. The measure consists of a series of developmental play tasks and takes and derives a developmental quotient (DQ) rather than an intelligence quotient (IQ). Raw scores of successfully completed items are converted to scale scores and to composite scores. These scores are used to determine the child's performance compared with norms taken from typically developing children of their age (in months). In one embodiment of the present invention, the language development in a subject is determined by a Bayley Scales of Infant and Toddler Development (BSID-IV).

The terms “supporting”, “promoting”, “enhancing” or “improving” may be used interchangeably in the context of the present invention. They should be understood as comprising supporting or helping the normal development or growth of an individual. The subject may not suffer from a disease but may be susceptible to the development of unhealthy conditions, for example later in life. For example, the one or more HMOs may be used to improve language development.

Within the context of the present invention, the expression “language development” refers to the process through which children acquire the ability to process speech and communicate.

Within the context of the present invention, the expression “nutritional composition” refers to a composition which nourishes a subject. This nutritional composition is usually taken orally or intravenously. It may include a lipid source or a fat source, a carbohydrate source and/or a protein source. Moreover, nutritional composition(s) may refer to liquids, powder, gels, pastes, solids, concentrates, suspensions, or ready-to-use forms of enteral formulas, oral formulas, formulas for infants, formulas for pediatric subjects, formulas for children, growing-up milks, porridges and/or infant cereals or baby food.

Within the context of the present invention, the expression “synthetic nutritional composition” refers to a mixture obtained by chemical and/or biological means that can be chemically identical to the mixture naturally occurring in mammalian milk (i.e. synthetic nutritional composition is not breast milk).

Within the context of the present invention, the terms “nutrient” or “nutrients” are intended to comprise both macronutrients (for example carbohydrates, proteins or fats) and micronutrients (for example minerals or vitamins) and component thereof (e.g., fatty acids, amino acids) for the human body.

Within the context of the present invention, the terms “ingredient” or “ingredients” indicate an edible substance or mixture of substances which comprises or is essentially consisting of a nutrient for the human body.

Within the context of the present invention the expression “infant formula” refers to a foodstuff intended for particular nutritional use by infants during the first months of life satisfying by itself the nutritional requirements of this category of person (Article 20 of the European Commission Directive 91/321/EEC 2006/EC of 22 Dec. 2006 on infant formulae and on follow-on formulae). It also refers to a nutritional composition intended for infants and as defined in Codex Alimentarius (Codex STAN 72-1981) and Infant Specialties (incl. Food For Special Medical Purpose). The expression “infant formula” encompasses both “starter infant formula” and “follow-up formula” or “follow-on formula”.

The expressions “follow-up formula” or “follow-on formula” is given from the 6 months onwards and includes growing-up milk. It constitutes the liquid element in the progressively diversified diet of this category of person.

The expression “baby food” means a foodstuff intended for particular nutritional use by infants or young children during the first year of life.

The expression “infant cereal composition” means a foodstuff intended for particular nutritional use by the infants or young children during the two years of life.

The expression “growing-up milk (or GUM)” refers to a milk-based drink generally with added vitamins and minerals that is intended for young children or children.

The expression “fortifier” refers to liquid or solid nutritional composition suitable for fortifying or mixing with human breast milk, infant formula, growing-up milk.

The expression “mother's milk” should be understood as the breast milk or the colostrum of the mother.

### Human Milk Oligosaccharides

The present invention relates to one or more human milk oligosaccharides (HMOs) for use in supporting brain development and/or cognitive development in a subject. In one embodiment, the one or more HMOs are used to support language development in infants or young children. In some embodiments, the one or more HMOs are used in a therapeutic method.

The advantage of the present invention is that the infants and young children follow a natural progression or timetable for mastering the skills of language in a period crucial for setting the important foundation for communicating and learning for the rest of child's life. Ensuring that the key milestones in language development are achieved may reduce the risk of delays in language development and resulting troubles in language. Supporting language development advantageously supports language mastership.

The one or more HMOs may be administered by any suitable route and in any suitable form. Suitably, the one or more HMOs are administered by oral and/or enteral administration. In preferred embodiments, the one or more HMOs are administered by oral administration. The one or more HMOs may be administered separately, simultaneously or sequentially. In preferred embodiments, the one or more HMOs are administered simultaneously.

Many different kinds of HMOs are found in human milk and are typically based on a combination of glucose, galactose, sialic acid (N-acetylneuraminic acid), fucose and/or N-acetylglucosamine with many and varied linkages between them. Almost all HMOs have a lactose moiety at their reducing end while sialic acid and/or fucose (when present) occupy terminal positions at the non-reducing ends. HMOs can be acidic (e.g. charged sialic acid containing oligosaccharides) or neutral (e.g. fucosylated oligosaccharides). See e.g. Chen, X., 2015. Advances in carbohydrate chemistry and biochemistry, 72, pp. 113-190.

HMOs found in human milk include Lacto-N-tetraose (LNT), Lacto-N-neotetraose (LNnT), 2′-Fucosyllactose (2′FL), 3-Fucosyllactose (3FL), Lactodifucotetraose (LDFT), Lacto-N-difucohexaose I (LNDFHI), Lacto-N-fucopentaose I (LNFP-I), Lacto-N-fucopentaose II (LNFP-II), Lacto-N-fucopentaose III (LNFP-III), Trifucosyllacto-N-hexaose (TF-LNH), 3′-Sialyllactose (3′SL), 6′-Sialyllactose (6′SL), Disialyllacto-N-tetraose (DSLNT), Sialyllacto-N-neotetraose c (LST c) and Fucosyldisialyllacto-N-hexaose I (FDS-LNH-I). See e.g., Soyyllmaz, B., et al., 2021. Nutrients, 13(8), p. 2737.

In some embodiments, the one or more HMOs comprise or consist of: 3-fucosyllactose (3FL), lacto-N-fucopentaose II (LNFPII), lacto-N-fucopentaose V (LNFPV), and/or any combination thereof.

The subject may be administered any suitable amounts of the one or more HMOs, in any suitable form and via any suitable route of administration (e.g., in any form and via any route described herein).

Suitable doses of HMOs are described in e.g., EFSA Panel on Nutrition, Novel Foods and Food Allergens (NDA), 2022. EFSA Journal, 20(5), p. e07257; EFSA Panel on Nutrition, Novel Foods and Food Allergens (NDA), 2021. EFSA Journal, 19(6), p.e06662; and EFSA Panel on Nutrition, Novel Foods and Food Allergens (NDA), 2022. EFSA Journal, 20(3), p.e07140.

The HMOs may be obtained by any suitable method. Suitable methods for synthesizing HMOs will be well known to those of skill in the art. For example, processes have been developed for producing oligosaccharides by microbial fermentations, enzymatic processes, chemical syntheses, or combinations of these technologies (see e.g., Zeuner et al., 2019. Molecules, 24(11), p. 2033).

### Fucosylated HMOs

In some embodiments, the one or more HMOs comprise or consist of a combination of fucosylated HMOs. In some embodiments, the one or more HMOs comprise or consist of fucosylated HMOs.

As used herein, fucosylated HMOs may refer to neutral HMOs which contain one or more fucose sugar. Fucosylated HMOs may be monofucosylated, difucosylated, or trifucosylated and preferably do not contain sialic acid. Non-limiting example(s) of fucosylated HMOs include: 2′-fucosyllactose (2′FL); 3-fucosyllactose (3FL); lactodifucotetraose (LDFT); lacto-N-fucopentaose, such as lacto-N-fucopentaose I (LNFP-I), lacto-N-fucopentaose II (LNFP-II), lacto-N-fucopentaose III (LNFP-III) or lacto-N-fucopentaose V (LNFP-V); lacto-N-fucohexaose, such as lacto-N-difucohexaose I (LNDFH-I) and lacto-N-difucohexaose II (LNDFH-II); lacto-N-neofucopentaose V (LNnFP-V); lacto-N-neodifucosylhexaose (LNnDFH), such as lacto-N-neodifucosylhexaose II (LNnDFH-II); fucosyllacto-N-hexaose (FLNH), such as fucosyllacto-N-hexaose 1 (FLNH-I) and fucosyllacto-N-hexaose II (FLNH-II); fucosyllacto-N-neohexaose (F-LNnH), such as fucosyllacto-N-neohexaose I (F-LNnH-I) and fucosyllacto-N-neohexaose II (F-LNnH-II); monofucosyllacto-N-hexaose-III (MFNLH-III); difucosyllacto-N-hexaose (DF-LNH); difucosyllacto-N-neohexaose (DF-LNnH), such as difucosyllacto-N-neohexaose I (DF-LNnH-I) and difucosyllacto-N-neohexaose II (DF-LNnH-II); difucosyllacto-N-hexaose-a (DFLNHa); trifucosyllacto-N-hexaose (TF-LNH); fucosyl-para-lacto-N-hexaose (F-pLNH); difucosyl-para-lacto-N-hexaose (DF-pLNH); tr-fucosyl-para-lacto-N-hexaose (TF-pLNH); and combinations thereof. See e.g., Chen, X., 2015. Advances in carbohydrate chemistry and biochemistry, 72, pp. 113-190.

In some embodiments, the one or more fucosylated HMOs comprise or consist of 3-fucosyllactose (3FL), lacto-N-fucopentaose II (LNFPII), lacto-N-fucopentaose V (LNFPV) and/or any combination thereof.

### 3-Fucosyllactose (3FL)

In some embodiments, the one or more HMOs comprise or consist of 3-fucosyllactose (3FL). In some embodiments, the one or more HMOs consist of 3FL. In some embodiments, the one or more HMOs are 3FL.

3-fucosyllactose (3-FL) is a fucosylated, neutral trisaccharide composed of L-fucose, D-galactose, and D-glucose units and may have the following structure:

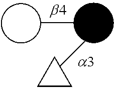

Where: ▴ is fucose (Fuc), ◯ is galactose, and is glucose.

Suitably, 3FL is administered to the subject in an amount of at least about 3.5 mg/L, at least about 3.75 mg/L, at least 4 mg/L, or at least 4.25 mg/L. Suitably, 3FL is administered to the subject in an amount of about 5,000 mg/L or less, about 4,000 mg/L or less, about 3′750 mg/L or less, or about 3′600 mg/L. Suitably, 3FL is administered to the subject in an amount of from about 3.5 mg/L to about 5,000 mg/L, from about 3.75 mg/L to about 4,000 mg/L, from about 4 mg/L to about 3′750 mg/L, or from about 4.25 mg/L to about 3′600 mg/L. In one embodiment, 3FL is administered to the subject in an amount of from about 4.25 mg/L to about 3′580 mg/L.

### Lacto-N-Fucopentaose II (LNFPII)

Lacto-N-fucopentaose II (LNFPII) is a fucosylated, non-sialylated Galβ1-3GlcNAc core (type 1 core) human milk oligosaccharide and may have the following structure:

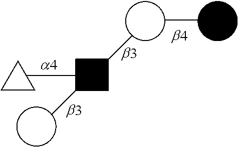

Where: ▴ is fucose (Fuc), ◯ is galactose, ● is glucose, and ▪ is N-acetylglucosamine (GlcNAc).

Suitably, LNFPII is administered to the subject in an amount of at least about 16 mg/L, at least about 16.5 mg/L, at least about 17 mg/L, at least about 17.5 mg/L. Suitably, LNFPII is administered to the subject in an amount of about 2,400 mg/L or less, about 2,350 mg/L or less, about 2,300 mg/L or less, or about 2,250 mg/L or less. Suitably, LNFPII is administered to the subject in an amount of from about 16 mg/L to about 2,400 mg/L, from about 16.5 mg/L to about 2,350 mg/L, from about 17 mg/L to about 2,300 mg/L, or from about 17.5 mg/L/to about 2,250 mg/L. In one embodiment, LNFPII is administered to the subject in an amount of from about 17.5 mg/L to about 2,250 mg/L.

### Lacto-N-Fucopentaose V (LNFPV)

Lacto-N-fucopentaose V (LNFPV) is a fucosylated, neutral pentasacchride Galβ1-3GlcNAcβ1-3Galβ1-4 (Fuca1-3)Glc core human milk oligosaccharide and may have the following structure:

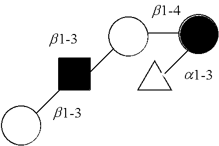

Where: ▴ is fucose (Fuc), ◯ is galactose, ● is glucose, and is N-acetylglucosamine (GlcNAc).

Suitably, LNFPV is administered to the subject in an amount of at least about 9 mg/L, at least about 10 mg/L, at least about 11 mg/L, at least about 12 mg/L. Suitably, LNFPV is administered to the subject in an amount of about, 450 mg/L or less, about 425 mg/L or less, about 400 mg/L or less, or about 375 mg/L or less. Suitably, LNFPV is administered to the subject in an amount of from about 9 mg/L to about 450 mg/L, from about 10 mg/L to about 425 mg/L, from about 11 mg/L to about 400 mg/L, or from about 12 mg/L to about 375 mg/L. In one embodiment, LNFPV is administered to the subject in an amount of from about 12 mg/L to about 375 mg/L.

### Nutritional Composition

Suitably, the one or more HMOs are in the form of a composition. The composition may comprise the one or more HMOs in any effective amount.

In one aspect, the present invention provides a composition (e.g., a nutritional composition such as an infant formula, a follow-on formula, or a growing-up milk) comprising one or more HMOs for use in supporting brain development and/or cognitive development in a subject. In one embodiment, the composition is used for supporting language development in a subject.

In another aspect, the present invention provides use of one or more HMOs for the manufacture of a composition (e.g., a nutritional composition such as an infant formula, a follow-on formula, or a growing-up milk) for supporting brain development and/or cognitive development in a subject. In one embodiment, the one or more HMOs are used for the manufacture of a composition for supporting language development in a subject.

In another aspect, the present invention provides a method for supporting brain development and/or cognitive development in a subject, wherein the method comprises administering a composition (e.g., a nutritional composition such as an infant formula, a follow-on formula, or a growing-up milk) comprising one or more HMOs. In one embodiment, the present invention relates to a method for supporting language development in a subject, wherein the method comprises administering a composition comprising one or more HMOs.

The composition can be any type of composition in which the one or more HMOs can be incorporated, such as a composition in the form of a food or a beverage product, a nutritional supplement, or a pharmaceutical composition. The composition may be in solid (e.g., powder), liquid or semi-liquid form. The one or more HMOs may be in the form of a food composition, a beverage, a nutritional formula, a nutritional supplement, a nutraceutical, or a pharmaceutical composition.

Food and beverage products include all products intended to be consumed orally by human beings, for the purpose of providing nutrition and/or pleasure. For example, the composition may be a food or a beverage product, such as for infants, toddlers and/or young children. Examples of food and beverage products include dairy products such as milk products or yogurts, soups, sauces, sweet and savoury snacks, powdered drinks and cereal products.

In some embodiments, the one or more HMOs are in the form of a nutritional composition, a medical food product for clinical nutrition, or a supplement.

In some embodiments, the one or more HMOs are in the form of a nutritional composition. As used therein a “nutritional composition” may mean a composition which nourishes a subject. This nutritional composition is usually to be taken orally or intravenously, and typically includes a lipid or fat source and a protein source.

The nutritional composition may be a synthetic nutritional composition. As used therein, a “synthetic composition” may mean a nutritional composition obtained by chemical and/or biological means that can be chemically substantially identical to the mixture naturally occurring composition.

In some embodiments, the one or more HMOs are in the form of a medical food product for clinical nutrition. As use therein, a “medical food product for clinical nutrition” may also be known as “Food for Special Medical Purposes (FSMP)” and refers to specialized food designed to help meet the nutritional or dietary needs of a subject living with a disease, disorder or medical condition who are temporarily or permanently unable to achieve an adequate nutritional intake from normal foods or through modification of the normal diet.

In some embodiments, the one or more HMOs are in the form of an infant formula, a follow-on formula, or a growing-up milk.

In some embodiments, the one or more HMOs are in the form of an infant formula. The term “infant formula” as used herein may refer to a foodstuff intended for particular nutritional use by infants during the first months of life and satisfying by itself the nutritional requirements of this category of person (see e.g., Article 2 (c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The infant formula can be a preterm infant formula, a human milk fortifier, a starter infant formula, a follow-on formula, a baby-food formula, or an infant cereal formula.

In some embodiments, the one or more HMOs are in the form of a starter infant formula. Typically, a “starter infant formula” is intended for infants from birth as a breast-milk substitute.

In some embodiments, the one or more HMOs are in the form of a follow-on formula. A “follow-on formula” or “follow-up formula” may be given from the 6th months onwards. It may constitute the principal liquid element in the progressively diversified diet of this category of person.

In some embodiments, the one or more HMOs are in the form of a preterm infant formula. The term “preterm infant formula” as used therein means an infant formula for a preterm infant.

In some embodiments, the one or more HMOs are in the form of a milk fortifier. The term “milk fortifier” as used herein refers to liquid or solid nutritional compositions suitable for mixing with breast milk (which is human milk for a human milk fortifier) or infant formula. It is used to increase the calories, protein, minerals and vitamins in breast milk fed to preterm infants or infants with a low birth weight. The term “breast milk” is to be understood as the mother's milk or the colostrum of the mother or a donor's milk or the colostrum of a donor's milk.

In some embodiments, the one or more HMOs are in the form of a baby-food formula. The term “baby food formula” as used herein means a foodstuff intended for particular nutritional use by infants or children such as young children, during the first years of life.

In some embodiments, the one or more HMOs are in the form of a growing-up milk. The term “growing-up milk” (or GUM) as used herein refers to a milk formula product given from one year onwards. It is generally a dairy-based beverage adapted for the specific nutritional needs of young children (e.g., children aged from about 1 to about 3). Growing-up milk may also be known as “young-child formula” or “toddlers' milk”.

In some embodiments, the one or more HMOs are in the form of an infant cereal composition. The term “infant cereal composition” as used herein refers to a foodstuff intended for particular nutritional use by infants or children such as young children, during the first years of life.

In some embodiments, the one or more HMOs are in the form of a milk formula. The term “milk formula” as used herein may refer to a foodstuff intended for e.g., childhood nutrition, which may provide the sole source or supplemental nutrition for children e.g. aged about 3 years or older. In some embodiments, the milk formula is a grow milk.

In some embodiments, the composition is in a powder form and reconstituted in an aqueous medium (e.g., water) prior to administration. In other embodiments, the composition is in a liquid form ready for administration (e.g., a ready-to-feed formula).

In another embodiment, the one or more HMOs are in the form of a supplement. As used herein, a “supplement” or “dietary supplement” may be used to complement the nutrition of a subject (it is typically used as such, but it might also be added to any kind of compositions intended to be ingested by the subject). When the composition is a supplement, it can be provided in the form of unit doses. Supplements are typically present in the form of a liquid, a gel, a powder or a tablet or capsule. Powder supplements typically encompass supplements to be dissolved in water or milk, or to be sprinkled on food or in a beverage. Such supplements are intended to provide additional nutrients and/or a health benefit to the subject consuming it. A supplement can be used for providing nutrients and/or a health benefit to human beings, as well as to animals.

In another embodiment, the one or more HMOs are in the form of a fortifier. The fortifier can be an infant formula fortifier or growing-up milk fortifier.

In another embodiment, the one or more HMOs are in the form of a pharmaceutical product. Pharmaceutical products include for example drops, syrups, powder, tablet or capsule products intended to treat or prevent an adverse medical condition in a subject in need thereof.

### Human Milk Oligosaccharides (HMOs)

The one or more HMOs may be present in the composition in any suitable amounts.

For example, in an infant formula, follow-on formula, or growing-up milk, the skilled person may base the amount of an HMO on the amount found in human breast milk produced for an infant or child of the same age, in particular by a nutritionally replete mother (see e.g., Soyyllmaz, B., et al., 2021. Nutrients, 13(8), p. 2737).

Suitably, 3FL is present in a total amount of from about 3.5 mg/L to about 5,000 mg/L, or from about 3.75 mg/L to about 4,000 mg/L, or from about 4 mg/L to about 3′750 mg/L, or from about 4.25 mg/L to about 3,600 mg/L of the nutritional composition according to the present invention. Suitably, 3FL is present in a total amount of from 2.7 g/100 g to 3,846 g/100 g, or from 2.9 g/100 g to 3,077 g/100 g, or from 3.1 g/100 g to 2,885 g/100 g, or from 3.3 g/100 g to 2,769 g/100 g of the nutritional composition according to the present invention.

Suitably, LNFPII is present in a total amount of from about 16 mg/L to about 2,400 mg/L, from about 16.5 mg/L to about 2,350 mg/L, from about 17 mg/L to about 2,300 mg/L, or from about 17.5 mg/L/to about 2,250 mg/L of the nutritional composition according to the present invention. Suitably, LNFPII is present in a total amount of from 12.3 g/100 g to 1,846 g/100 g, or from 12.7 g/100 g to 1,808 g/100 g, or from 13.1 g/100 g to 1,769 g/100 g, or from 13.5 g/100 g to 1,731 g/100 g of the nutritional composition according to the present invention.

Suitably, LNFPV is present in a total amount of from about 9 mg/L to about 450 mg/L, from about 10 mg/L to about 425 mg/L, from about 11 mg/L to about 400 mg/L, or from about 12 mg/L to about 375 mg/L of the nutritional composition according to the present invention. Suitably, LNFPV is present in a total amount of from 6.9 g/100 g to 346.2 g/100 g, or from 7.7 g/100 g to 327 g/100 g, or from 8.5 g/100 g to 308 g/100 g, or from 9.2 g/100 g to 288.5 g/100 g of the nutritional composition according to the present invention.

However, the one or more HMOs present in the nutritional composition may be used outside the depending on for example bioavailability of said HMOs from infant formula in comparison to human breast milk.

### Other Components

A nutritional composition (e.g., infant formula, follow-on formula, growing-up milk) of the present invention may contain a protein source, a carbohydrate source and a lipid source. In some embodiments however, especially if a nutritional composition of the invention is a supplement or a fortifier, there may be only lipids (or a lipid source).

The nutritional composition (e.g., infant formula, follow-on formula, growing-up milk) of the present invention may comprise from about 50 kcal/100 mL to about 90 kcal/100 ml, from about 55 kcal/100 mL to about 85 kcal/100 ml, from about 60 kcal/100 mL to about 80 kcal/100 ml, from about 65 kcal/100 mL to about 75 kcal/100 ml, or from about 65 kcal/100 mL to about 70 kcal/100 ml. If the nutritional composition is in the form of a powder these amounts may be following reconstitution with liquid according to the instructions provided.

The nutritional composition (e.g., infant formula, follow-on formula, or growing-up milk) of the invention may comprise about 100 kcal/100 g to about 1000 kcal/100 g, about 200 kcal/100 g to about 800 kcal/100 g, or about 400 kcal/100 g to about 600 kcal/100 g, on a dry weight basis.

### Protein

A nutritional composition (e.g., infant formula, follow-on formula, or growing-up milk) according to the invention may contain a protein source. The protein may be in an amount of from about 1 g to about 4 g per 100 kcal, about 1.5 g to about 3 g per 100 kcal, or about 1.5 g to about 2.5 g per 100 kcal.

Protein sources based on, for example, whey, casein and mixtures thereof may be used as well as plant-based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e., more than 50% of proteins are coming from whey proteins, such as more than 60% or more than 70%). The proteins may be intact or hydrolysed or a mixture of intact and hydrolysed proteins. By the term “intact” it is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example, about 7% by weight of lysine which greatly improves the nutritional quality of the protein source.

In one particular embodiment, the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90.

At least 70%, 80%, 85%, 90%, 95% or 97% of the proteins may be hydrolysed. In a particular embodiment, 100% of the proteins are hydrolysed.

In one particular embodiment, the proteins of the composition are plant-based protein.

### Carbohydrate

A nutritional composition (e.g., infant formula, follow-on formula, or growing-up milk) according to the present invention may contain a carbohydrate source. The carbohydrate may be in an amount of from about 5 g to about 20 g per 100 kcal, about 10 g to about 15 g per 100 kcal, about 8 g to about 15 g per 100 kcal.

Any carbohydrate source conventionally found in nutritional compositions such as lactose, sucrose, saccharose, maltodextrin, starch and mixtures thereof may be used although one of the preferred sources of carbohydrates is lactose.

### Lipid

A nutritional composition (e.g., infant formula, follow-on formula, or growing-up milk) according to the present invention may contain lipids and essential fatty acids. The lipids may be in an amount of from about 1 g to about 10 g per 100 kcal, about 2 g to about 8 g per 100 kcal, or about 3 g to about 8 g per 100 kcal.

Non-limiting examples of lipids include: palm olein, high oleic sunflower oil, high oleic safflower oil, canola oil, fish oil, coconut oil, bovine milk fat, and combinations thereof. It may be particularly beneficial if the composition comprises fat in an amount of about 25 to about 30 g/100 g dry weight of the composition. Non-limiting examples of essential fatty acids include: linoleic acid (LA), a-linolenic acid (ALA). Compositions of the invention may further contain gangliosides monosialoganglioside-3 (GM3) and disialogangliosides 3 (GD3), and combinations thereof.

### Others

A nutritional composition (e.g., infant formula, follow-on formula, or growing-up milk) of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B3, vitamin B6, vitamin B12, vitamin E, vitamin K1, vitamin K2, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, a nutritional composition (e.g., infant formula, follow-on formula, or growing-up milk) of the invention may contain emulsifiers and stabilisers such as soy, lecithin, citric acid esters of mono- and diglycerides, and the like.

### Preparation of Compositions

The compositions according to the present invention may be prepared by any known or otherwise suitable manner. For example, a nutritional composition (e.g. infant formula, follow-on formula, or growing-up milk) may be proposed by blending together a source of protein with a carbohydrate source and a lipid source in appropriate proportions. If used, emulsifiers may be included at this stage. Vitamins and minerals may be added at this stage but may also be added later to avoid thermal degradation. Water, preferably water which has been subjected to reverse osmosis or deionized water, may then be added and mixed in to form a liquid mixture. The temperature of mixing is preferably room temperature but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formula are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula. Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid formula, the homogenized mixture may be filled into suitable containers, preferably aseptically. However, the liquid composition may also be retorted in the container, suitable apparatus for carrying out the filling and retorting of this nature is commercially available.

Method for supporting brain and/or cognitive development The one or more HMOs may be used to support brain and/or cognitive development.

As used therein, “supporting brain and/or cognitive development” may refer to the support of normal brain and/or cognitive development in a subject, for example during infancy or childhood. Supporting brain and/or cognitive development may support timely development of cognitive skills.

In one aspect, the present invention provides one or more HMOs for use in supporting brain and/or cognitive development in a subject. In another aspect, the present invention provides use of one or more HMOs for supporting brain and/or cognitive development in a subject. In another aspect, the present invention provides a method for supporting brain and/or cognitive development in a subject, wherein the method comprises administering to the subject an effective amount of one or more HMOs.

In one aspect, the present invention provides a combination of one or more fucosylated HMOs for use in supporting brain and/or cognitive development in a subject. In another aspect, the present invention provides use of one or more fucosylated HMOs for supporting brain and/or cognitive development in a subject. In another aspect, the present invention provides a method for supporting brain and/or cognitive development in a subject, wherein the method comprises administering to the subject an effective amount of one or more fucosylated HMOs. Suitably, 3-fucosyllactose (3FL) is administered in an amount of from about 3.5 mg/L to about 5,000 mg/L, lacto-N-fucopentaose II (LNFPII) is administered in an amount of from about 16 mg/L to about 2,400 mg/L and lacto-N-fucopentaose V (LNFPV) is administered in an amount of from about 9 mg/L to about 450 mg/L.

In one aspect, the present invention provides 3FL for use in supporting brain and/or cognitive development in a subject. In another aspect, the present invention provides use of 3FL for supporting brain and/or cognitive development in a subject. In another aspect, the present invention provides a method for supporting brain and/or cognitive development in a subject, wherein the method comprises administering an effective amount of 3FL to the subject.

In one aspect, the present invention provides LNFPII for use in supporting brain and/or cognitive development in a subject. In another aspect, the present invention provides use of LNFPII for supporting brain and/or cognitive development in a subject. In another aspect, the present invention provides a method for supporting brain and/or cognitive development in a subject, wherein the method comprises administering an effective amount of LNFPII to the subject.

In one aspect, the present invention provides LNFPV for use in supporting brain and/or cognitive development in a subject. In another aspect, the present invention provides use of LNFPV for supporting brain and/or cognitive development in a subject. In another aspect, the present invention provides a method for supporting brain and/or cognitive development in a subject, wherein the method comprises administering an effective amount of LNFPV to the subject.

The one or more HMOs according to the present invention may support brain and/or cognitive development of a subject at 0, 1, 2, 3, 4, 5, 6, 9, 12, 18, 24 or 36 months.

### Method for Supporting Language Development

The one or more HMOs may be used to support language development. As used therein, “supporting language development” may refer to the support of normal language development in a subject. In particular, it is important that the milestones of language development are achieved following a natural progression or timetable in order to avoid delays in language development that may eventually result in language disorder.

In one aspect, the present invention provides one or more HMOs for use in supporting language development in a subject. In another aspect, the present invention provides use of one or more HMOs for supporting language development in a subject. In another aspect, the present invention provides a method for supporting language development in a subject, wherein the method comprises administering to the subject an effective amount of one or more HMOs.

In one aspect, the present invention provides a combination of one or more fucosylated HMOs for use in supporting language development in a subject. In another aspect, the present invention provides use of one or more fucosylated HMOs for supporting language development in a subject. In another aspect, the present invention provides a method for supporting language development in a subject, wherein the method comprises administering to the subject an effective amount of one or more fucosylated HMOs. Suitably, 3-fucosyllactose (3FL) is administered in an amount of from about 3.5 mg/L to about 5,000 mg/L, lacto-N-fucopentaose II (LNFPII) is administered in an amount of from about 16 mg/L to about 2,400 mg/L and lacto-N-fucopentaose V (LNFPV) is administered in an amount of from about 9 mg/L to about 450 mg/L.

In one aspect, the present invention provides 3FL for use in supporting language development in a subject. In another aspect, the present invention provides use of 3FL for supporting language development in a subject. In another aspect, the present invention provides a method for supporting language development in a subject, wherein the method comprises administering an effective amount of 3FL to the subject.

In one aspect, the present invention provides LNFPII for use in supporting language development in a subject. In another aspect, the present invention provides use of LNFPII for supporting language development in a subject. In another aspect, the present invention provides a method for supporting language development in a subject, wherein the method comprises administering an effective amount of LNFPII to the subject.

In one aspect, the present invention provides LNFPV for use in supporting language development in a subject. In another aspect, the present invention provides use of LNFPV for supporting language development in a subject. In another aspect, the present invention provides a method for supporting language development in a subject, wherein the method comprises administering an effective amount of LNFPV to the subject.

The one or more HMOs according to the present invention may support brain and/or cognitive development of a subject at 0, 1, 2, 3, 4, 5, 6, 9, 12, 18, 24 or 36 months.

### Subject

The subject may be any suitable subject. Suitably, the subject may be a human.

In preferred embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 10 years, from about 3 years to about 9 years, from about 3 years to about 8 years, from about 3 years to about 7 years, from about 3 years to about 6 years, or from about 3 years to about 5 years.

In preferred embodiments, the subject is an infant. In some embodiments, the subject is a newborn infant. In some embodiments, the subject is a preterm infant.

In some embodiments, the subject is aged about 5 years or less, about 4 years or less, about 3 years or less, about 2 years or less, or about 1 year or less. In some embodiments, the subject is aged about 12 months or less, about 11 months or less, about 10 months or less, about 9 months or less, about 8 months or less, about 7 months or less, or about 6 months or less. In some embodiments, the subject is aged about 6 months or less.

In some embodiments, the one or more HMOs are administered during the first 1, 2, 3,4, 5, 6, 7, 8, 9, 10, 11, 12, 18, 14, or 36 months of life of a subject. For example, the one or more HMOs are administered to a subject starting from birth up to 1, 2, 3,4, 5, 6, 7, 8, 9, 10, 11, 12, 18, 24 or 36 months.

The subject may be at risk of inadequate brain and/or cognitive development. Major risks for poor child development include intrauterine growth restriction, stunting, iodine deficiency, iron-deficiency anemia, malaria, lead exposure, HIV, maternal depression, and inadequate cognitive stimulation. Additionally, maternal education and breastfeeding have been identified as protective factors (Walker, S. P., et al., 2011. The Lancet, 378(9799), pp. 1325-1338). In some embodiments, the subject has and/or has suffered from intrauterine growth restriction, stunting, iodine deficiency, iron-deficiency anemia, malaria, lead exposure, HIV, maternal depression, and/or inadequate cognitive stimulation. The subject may be at risk of inadequate language development.

The subject may have suffered from and/or may be suffering from inadequate brain and/or cognitive development. The subject may be and/or may suffer from inadequate language development. This may be determined by using any method known in the art, for example by using Bayley Scales of Infant and Toddler Development (BSID-IV).

In one embodiment, the subject's language development is determined by a Bayley Scales of Infant and Child Development (BSID-IV)

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

Example-Association of brain development with breast milk concentrations of human milk oligosaccharides (HMOs)

The invention is further described with reference to the following example. It will be appreciated that the invention as claimed is not intended to be limited in any ways by these examples.

The inventors investigated the association of brain and/or cognitive development with breast milk concentrations of specific HMOs in human breast milk.

### Study Design

Children for this study were selected from the observational breastfeeding arm of a prospective longitudinal randomized control trial. Infants for the trial were recruited and followed at two study sites in the US (Rhode Island Hospital in Providence, RI, and Pennington Biomedical Research Center, in Baton Rouge, LA), with seven study visits conducted at V1 (6±1 week of life), V2 (3 months±2 weeks), V3 (6 months±2 weeks), V4 (9 months±2 weeks), and V5 (12 months±2 weeks), V6 (18 months±3 weeks), V7 (24 months±4 weeks). For the observational breastfeeding arm, mothers and their infants were recruited at 6 weeks following delivery and were included if more than 90% of nutritional intake and no infant formula or solids for more than 10% of the nutritional intake through at least 4 months of life.

Other specific inclusion criteria included: English speaking mothers at least 18 years of age with a maternal intelligence quotient (IQ, Wechsler Adult Intelligence Scale) of at least 70 healthy pregnancy and uncomplicated delivery at 38-41 weeks gestation, birth weight >2000 g, and use of any unsafe psychopharmacological treatment by the mother during pregnancy or lactation, including medical treatment of depression.

In total, 107 mother-child dyads were enrolled (61 female) HMO data were available for N=108, 97, 76 individuals at 2-5 weeks, 6 weeks, 3 months (visits V0-V2), respectively.

Breast milk samples were collected from mothers longitudinally at the V0, V1, and V2 study visits using a hospital grade electric breast pump and a full expression sample was collected. Samples were collected between 10 AM-12PM from the right breast. Mothers were asked to empty the right breast approximately 2 hours prior to milk sampling.

### Cognitive Assessments

Infant cognitive was assessed using the Bayley Scales of Infant and Toddler Development, 4th edition (BSID-IV) at 6, 12 and 24 months.

TABLE I
Summary statistics for BSID-IV by time point

| Scale | Visit | | Mean (SD) | Median [Min, Max] |
|---|---|---|---|---|
| BSID: Language | 6 mo | (N = 77) | 19.4 (3.75) | 19.0 [12.0, 26.0] |
| | 12 mo | (N = 53) | 17.8 (3.84) | 18.0 [11.0, 24.0] |
| | 24 mo | (N = 51) | 18.1 (4.31) | 18.0 [6.00, 26.0] |
| BSID: Cognition | 6 mo | (N = 76) | 10.1 (2.21) | 11.0 [6.00, 15.0] |
| | 12 mo | (N = 53) | 10.0 (1.99) | 10.0 [6.00, 15.0] |
| | 24 mo | (N = 51) | 10.2 (1.95) | 10.0 [4.00, 14.0] |
| BSID: Motor dev. | 6 mo | (N = 76) | 19.0 (4.43) | 19.0 [8.00, 29.0] |
| | 12 mo | (N = 53) | 18.8 (2.86) | 19.0 [12.0, 25.0] |
| | 24 mo | (N = 51) | 18.1 (3.18) | 18.0 [12.0, 24.0] |

### Human Milk Oligosaccharides (HMOs)

23 HMOs (Table II) were analyzed by ultra-high performance liquid chromatography with fluorescence detection (UHPLC-FLD), according to the method of Austin and Benet (21). 2,FL, 3FL, 3,SL, 6,SL, LNT, LNnT and LNFP-I were quantified against genuine standards of analytical quality, all other HMOs were quantified against maltotriose assuming equimolar response factors.

TABLE II
HMOs measured with their abbreviations

| HMO | Abbreviation |
|---|---|
| 2′-Fucosyllactose | 2′FL |
| 3′-Galactosyllactose | 3′GL |
| 3′-Sialyllactose | 3′SL |
| 3,2′-Difucosyllactose | DFSL |
| 3-Fucosyllactose | 3FL |
| 6′-Galactosyllactose | 6′GL |
| 6′-Sialyllactose | 6′SL |
| Difucosyllacto-N-hexaose-A | DFNHA |
| Disialyllacto-N-tetraose | DSLNT |
| Hex4 HexNAc2 (2) | HEX |
| Lacto-N-difucohexaose-I | LNDFI |
| Lacto-N-fucopentaose-I | LNFPI |
| Lacto-N-fucopentaose-Il | LNFPII |
| Lacto-N-fucopentaose-III | LNFPIII |
| Lacto-N-fucopentaose-V | LNFPIV |
| Lacto-N-hexaose | LNH |
| Lacto-N-neodifucohexaose | LNnDFH |
| Lacto-N-neofucopentaose-V | LNnFPV |
| Lacto-N-neotetraose | LNnT |
| Lacto-N-tetraose | LNT |
| Monofucosyllacto-N-hexaose-III | MFLNIII |
| Sialyllacto-N-tetraose-b | LSTb |
| Sialyllacto-N-tetraose-c | LSTc |

### Statistical Analysis

Spearman correlations with a Benjamini-Hochsberg correction were used to investigate the direct pairwise associations HMOs and cognitive measures (BSID-IV). We analyzed the three BSID-IV subscales (cognition, language and motor development).

All analyses were performed with R, version 4.0.2

### Correlations Between Breastmilk HMO Concentrations and BSID-IV Scores

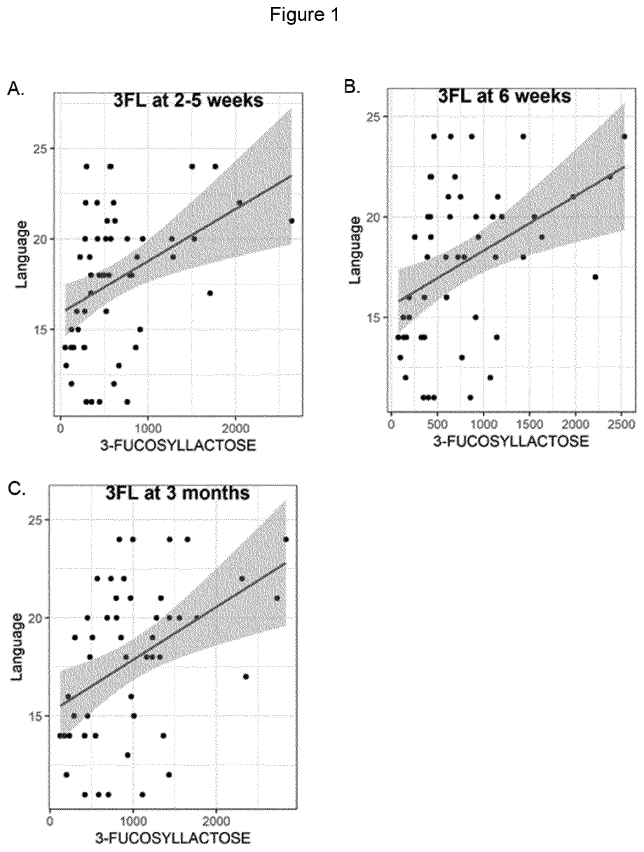

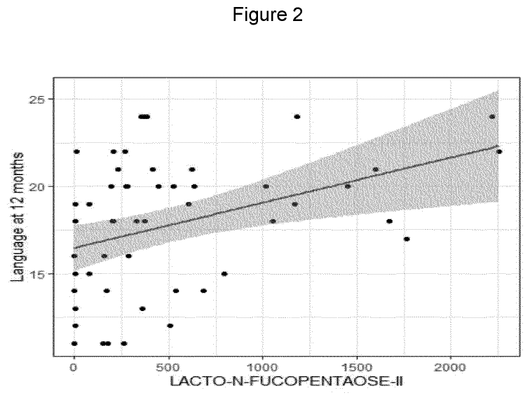

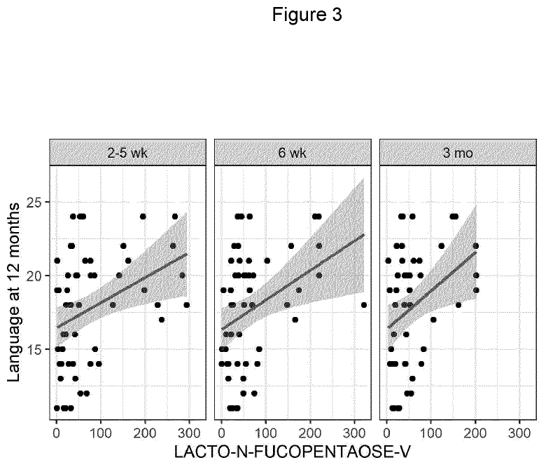

Cross-sectional correlations between the concentrations of HMOs and the BSID-IV scores are summarized in Table III, with significant correlations observed between Language at 12 months and 3FL, LNFPII, LNFPV concentrations at 2-5 weeks (FIG. 1, FIG. 2 and FIG. 3). 3FL was also significantly correlated also at 6 weeks and 3 months (FIG. 1).

TABLE III
Correlations between HMO concentrations
(mg/L) and Bayley (after B-H correction)

| | | | Time point | | | | |
|---|---|---|---|---|---|---|---|
| | HMO | | Milk | | Timepoint | | p. |
| Correlation | [mg/L] | outcome | sample | | Bayley | N | adj. |
| 0.42 | 3FL | Language | 2-5 | weeks | 12 months | 54 | 0.02 |
| 0.45 | 3FL | Language | 6 | weeks | 12 months | 51 | 0.02 |
| 0.44 | 3FL | Language | 2-5 | weeks | 12 months | 48 | 0.04 |
| 0.43 | LNFPII | Language | 2-5 | weeks | 12 months | 54 | 0.02 |
| 0.41 | LNFPV | Language | 2-5 | weeks | 12 months | 54 | 0.02 |

Child's sex was associated with language (functional ANOVA, p=0.047), with males having an average score of 17.0 across all visits, compared to an average score of 19.3 for females.

The positive association between language at 12 months and 3-FL remained significant also after adjusting for gender and gestational age (Table IV).

TABLE IV
Linear model for language at 12 months, with 3FL as independent
variable, adjusted for gender and gestational age

| term | estimate | std. error | p. value |
|---|---|---|---|
| (Intercept) | −28.45 | 9.87 | 0.00 |
| 3FL | 18.87 | 6.60 | 0.00 |
| Gestational Age | 1.16 | 0.25 | 0.00 |
| SEX: Male | −1.57 | 0.59 | 0.01 |
| VISIT: V1 | −0.23 | 1.03 | 0.83 |
| VISIT: V2 | −0.31 | 1.07 | 0.77 |
| 3FL: VISIT V1 | −3.92 | 8.39 | 0.64 |
| 3FL: VISIT V2 | −8.67 | 7.58 | 0.25 |

While receptive language capabilities emerge in the first 12 months of infancy due to rapid maturation of auditory functions (McGurk et al., Percept Psychophys. 1978; 24(3):253-7), expressive language capabilities emerge at or post 12 months of age (Scharf et al., Pediatr Rev. 2016; 37(1):25-37). Thus 12 months can be considered as a pivotal age for the development of language capabilities in infants. In terms of existing knowledge on the association between HMOs and language development in infancy, Cho et al., (Am J Clin Nutr 2021; 114:588-597) were the first to report an association between syalilated HMO 3′SL and language development. More specifically, Cho et al., found a stronger association between 3′SL and receptive language scores at 12 months. Contrary to these findings, we found several significant associations between a specific group of fucosyllated HMOs (3 FL, LNFP II, LNFP V) and language scores in 12-month-old infants. While the concentrations of 3FL collected at 2-5 weeks, 6 weeks and 3 months from HM positively correlated with language scores at 12 months, the concentrations LNFP-II and LNFP-V collected at 2-5 weeks from HM positively correlated with language scores in 12-month-old infants.

### Embodiments

Various preferred features and embodiments of the present invention will now be described with reference to the following numbered paragraphs (paras).
1. One or more human milk oligosaccharides (HMOs) for use in supporting brain development and/or cognitive development in a subject, wherein the one of more HMOs comprise or consist of: fucosyllactose (3FL), lacto-N-fucopentaose II (LNFPII), lacto-N-fucopentaose V (LNFPV), and/or any combination thereof.
2. The one or more HMOs for use according to para 1, wherein the HMOs support language development in a subject.
3. The one or more HMOs for use according to para 1 or 2, wherein the subject's language development is determined by a Bayley Scales of Infant and Child Development (BSID-IV).
4. The one or more HMOs for use according to any of paras 1-3, wherein the one or more HMOs are administered by oral administration.
5. The one or more HMOs for use according to any of paras 1-4, wherein the one or more HMOs are administered separately, simultaneously or sequentially, preferably wherein the one or more HMOs are administered simultaneously.
6. The one or more HMOs for use according to any of paras 1-5, wherein fucosyllactose (3FL) is administered to the subject in an amount of from about 3.5 mg/L to about 5,000 mg/L, lacto-N-fucopentaose II (LNFPII) is administered to the subject in an amount of from about 16 mg/L to about 2,400 mg/L and/or lacto-N-fucopentaose V (LNFPV) is administered to the subject in an amount of from about 9 mg/L to about 450 mg/L.
7. The one or more HMOs for use according to any of paras 1-6, wherein the one or more HMOs are in the form of a nutritional composition.
8. The one or more HMOs for use according to para 7, wherein the nutritional composition is an infant formula, a follow-on formula, or a growing-up milk, preferably wherein the nutritional composition is an infant formula.
9. The one or more HMOs for use according to para 7 or 8, wherein the nutritional composition comprises fucosyllactose (3FL) in a total amount of from about 3.5 mg/L to about 5,000 mg/L, lacto-N-fucopentaose II (LNFPII) in an amount of from about 16 mg/L to about 2,400 mg/L and/or lacto-N-fucopentaose V (LNFPV) in an amount of from about 9 mg/L to about 450 mg/L.
10. The one or more HMOs according to any of paras 1-9, wherein the subject is a human subject.
11. The one or more HMOs according to any of paras 1-10, wherein the subject is an infant, a toddler or a young child, preferably an infant.
12. The one or more HMOs according to any of paras 1-11, wherein the subject is aged about 12 months or younger, about 11 months or younger, about 10 months or younger, about 9 months or younger, about 6 months or younger, about 3 months or younger, or about 1 months or younger.
13. The use of one or more HMOs for supporting brain development and/or cognitive development in a subject, the one or more HMOs comprising or consisting of: fucosyllactose (3FL), lacto-N-fucopentaose II (LNFPII), lacto-N-fucopentaose V (LNFPV) and/or any combination thereof.
14. The use of HMOs according to para 13, wherein the HMOs support language development in a subject.
15. The use of HMOs according to para 12 or 13, wherein the subject's language development is determined by a Bayley Scales of Infant and Child Development (BSID-IV).
16. The use of HMOs according to any of paras 13-15, wherein the one or more HMOs are administered by oral administration.
17. The use of HMOs according to any of paras 13-16, wherein the one or more HMOs are administered separately, simultaneously or sequentially, preferably wherein the one or more HMOs are administered simultaneously.
18. The use of HMOs according to any of paras 13-17, wherein fucosyllactose (3FL) is administered to the subject in an amount of from about 3.5 mg/L to about 5,000 mg/L, lacto-N-fucopentaose II (LNFPII) is administered to the subject in an amount of from about 16 mg/L to about 2,400 mg/L and/or lacto-N-fucopentaose V (LNFPV) is administered to the subject in an amount of from about 9 mg/L to about 450 mg/L.
19. The use of HMOs according to any of paras 13-18, wherein the one or more HMOs are in the form of a nutritional composition.
20. The use of HMOs according to para 19, wherein the nutritional composition is an infant formula, a follow-on formula, or a growing-up milk, preferably wherein the nutritional composition is an infant formula.
21. The use of HMOs according to para 19 or 20, wherein the nutritional composition comprises fucosyllactose (3FL) in a total amount of from about 3.5 mg/L to about 5,000 mg/L, lacto-N-fucopentaose II (LNFPII) in an amount of from about 16 mg/L to about 2,400 mg/L and/or lacto-N-fucopentaose V (LNFPV) in an amount of from about 9 mg/L to about 450 mg/L.
22. The use of HMOs according to any of paras 13-21, wherein the subject is a human subject.
23. The use of HMOs according to any of paras 13-22, wherein the subject is an infant, a toddler or a young child, preferably an infant.
24. The use of HMOs according to any of paras 19-23, wherein the subject is aged about 12 months or younger, about 11 months or younger, about 10 months or younger, about 9 months or younger, about 6 months or younger, about 3 months or younger, or about 1 months or younger.
25. A method for supporting brain and/or cognitive development in a subject, comprising administering to the subject an effective amount of one or more human milk oligosaccharides (HMOs), wherein the one or more HMOs comprise or consist of: fucosyllactose (3FL), lacto-N-fucopentaose II (LNFPII), lacto-N-fucopentaose V (LNFPV) and/or any combination thereof.
26. The method according to para 25, wherein the HMOs support language development in a subject.
27. The method according to para 25 or 26, wherein the subject's language development is determined by a Bayley Scales of Infant and Child Development (BSID-IV).
28. The method according to any of paras 25-27, wherein the one or more HMOs are administered by oral administration.
29. The method according to any of paras 25-28, wherein the one or more HMOs are administered separately, simultaneously or sequentially, preferably wherein the one or more HMOs are administered simultaneously.
30. The method according to any of paras 25-29, wherein fucosyllactose (3FL) is administered to the subject in an amount of from about 3.5 mg/L to about 5,000 mg/L, lacto-N-fucopentaose II (LNFPII) is administered to the subject in an amount of from about 16 mg/L to about 2,400 mg/L and/or lacto-N-fucopentaose V (LNFPV) is administered to the subject in an amount of from about 9 mg/L to about 450 mg/L.
31. The method according to any of paras 25-30, wherein the one or more HMOs are in the form of a nutritional composition.
32. The method according to para 31, wherein the nutritional composition is an infant formula, a follow-on formula, or a growing-up milk, preferably wherein the nutritional composition is an infant formula.
33. The method according to para 31 or 32, wherein the nutritional composition comprises fucosyllactose (3FL) in a total amount of from about 3.5 mg/L to about 5,000 mg/L, lacto-N-fucopentaose II (LNFPII) in an amount of from about 16 mg/L to about 2,400 mg/L and/or lacto-N-fucopentaose V (LNFPV) in an amount of from about 9 mg/L to about 450 mg/L.
34. The method according to any of paras 25-33, wherein the subject is a human subject.
35. The method according to any of paras 25-34, wherein the subject is an infant, a toddler or a young child, preferably an infant.
36. The method according to any of paras 25-35, wherein the subject is aged about 12 months or younger, about 11 months or younger, about 10 months or younger, about 9 months or younger, about 6 months or younger, about 3 months or younger, or about 1 months or younger.

1. A method of supporting brain development and/or cognitive development in a subject, the method comprising administering one or more human milk oligosaccharides (HMOs) to the subject, wherein the one of more HMOs comprise at least one of fucosyllactose (3FL), lacto-N-fucopentaose II (LNFPII), or lacto-N-fucopentaose V (LNFPV).

2. The method according to claim 1, wherein the one or more HMOs support language development in the subject.

3. The method according to claim 1, wherein language development of the subject is determined by a Bayley Scales of Infant and Toddler Development (BSID-IV).

4. The method according to claim 1, wherein the one or more HMOs are administered by oral administration to the subject.

5. The method according to claim 1, wherein the one or more HMOs are administered separately, simultaneously or sequentially.

6. The method according to claim 1, wherein fucosyllactose (3FL) is administered to the subject in an amount from about 3.5 mg/L to about 5,000 mg/L, lacto-N-fucopentaose II (LNFPII) is administered to the subject in an amount from about 16 mg/L to about 2,400 mg/L and/or lacto-N-fucopentaose V (LNFPV) is administered to the subject in an amount from about 9 mg/L to about 450 mg/L.

7. The method according to claim 1, wherein the one or more HMOs are administered in a nutritional composition.

8. The method according to claim 7, wherein the nutritional composition is an infant formula, a follow-on formula, or a growing-up milk.

9. The method according to claim 1, wherein the subject is an infant, a toddler or a young child.

10. The method according to claim 1, wherein the subject is aged about 12 months or younger.

11-15. (canceled)

16. The method according to claim 1, wherein the one or more human milk oligosaccharides comprise at least two of 3FL, LNFPII or LNFPV.

17. The method according to claim 1, wherein the one or more human milk oligosaccharides comprise all three of 3FL, LNFPII or LNFPV.

18. The method according to claim 1, wherein the one or more HMOs are administered simultaneously.

19. The method according to claim 1, wherein the nutritional composition is an infant formula.

20. The method according to claim 1, wherein the subject is an infant.
